(19) United States
(12) Patent Application Publication
(10) Pub. No.: US 2009/0076468 A1
(43) Pub. Date: Mar. 19, 2009

(54) PORTABLE LAVAGE

(76) Inventor: Peter Orange Hoffoss, Lino Lakes, MN (US)

Correspondence Address: ITALIA IP, 3500 WEST OLIVE AVE., SUITE 300, BURBANK, CA, 91505, US

(21) Appl. No.: 12/206,294

(22) Filed: Sep. 8, 2008

Related U.S. Application Data
(60) Provisional application No. 60/972,153, filed on Sep. 13, 2007.

(51) Int. Cl. A61M 3/02

(52) U.S. Cl. 604/310

(57) ABSTRACT

A liquid dispenser for enabling personal lavage, comprising a housing containing a see-through liquid reservoir, a pump, and a rechargeable battery. Liquids discharged from the pump pass through a flexible conduit which for at least part of its length is routed outside the housing. The housing may include a clip for holding the flexible conduit. The flexible conduit terminates in a form holding nozzle which may have a removable nozzle cover for discharging liquids, and a controller. The controller may comprise a resistor type switch arranged to vary pump speed, or may comprise a wireless signal generator. In the latter case, the liquid dispenser includes a transducer adapted to respond to wireless signals to control the pump. Both the form holding nozzle and its removable nozzle cover may be readily removed and replaced. A lamp may signal low battery voltage.

### CROSS-REFERENCE TO RELATED APPLICATION

This application claims the benefit of the filing date under 35 USC 119(e) of the filing date of U.S. Provisional Application Ser. No. 60/972,153, filed Sep. 13, 2007, the contents of which are hereby incorporated by reference.

### FIELD OF THE INVENTION

The present invention relates to an appliance for personal lavage, and more particularly to a self-powered dispenser of wash water.

### BACKGROUND OF THE INVENTION

Personal hygiene may require lavage in the anal and genital areas of the body. While this may be attended to routinely in showers and baths, there arise situations when lavage is required, and showers and baths are unavailable or not feasible. For example, those who have hemorrhoids would benefit from lavage, as would individuals with pH issues in that an additive such as baking soda could be available to moderate pH of bodily secretions. Nurses in maternity wards and midwives at homes would benefit from ability to dispense water containing Epsom salts.

A bidet may be employed to address the situations discussed above. However, a bidet is typically sufficiently large and unwieldy to be portable, and requires supply and waste plumbing connections. A bidet may present objections in that it is relatively expensive, and competes for precious floor space with other bathroom facilities, such as tubs, shower stalls, sinks, and toilets in most residential spaces.

There exists a need for a portable appliance which provides the function of a bidet, and which enables additives to be easily mixed with water to be dispensed.

### SUMMARY OF THE INVENTION

The present invention answers the above need by providing a relatively compact, portable, inexpensive appliance which assists in personal lavage. The novel appliance comprises a tank or reservoir for holding sufficient water to conduct personal lavage, a self-contained pressure source, and may have a flexible dispensing hose terminating in a convenient solid or rigid hand held dispenser. The pressure source may comprise a pump and a rechargeable battery. Appropriate controls may be provided to control the pump.

The novel appliance may be considerably smaller, lighter, less expensive, and portable than a bidet or other plumbing fixture. It may be easily filled with water and provided with a desired additive. It may be rested on the floor of a building when being used, so that the user need only hold the dispenser. The water reservoir may advantageously be relatively tall compared to width and depth when placed on the floor, so that it occupies relatively little floor space.

It is an object of the invention to provide an appliance capable of performing many of the functions of a bidet, but which is relatively compact, lightweight, and inexpensive, compared to a bidet, and which is portable.

Another object of the invention is to enable lavage water to be mixed with additives.

A further object of the invention is to enable direction of discharge of water by a hand wielded device.

An additional object of the invention is to enable the weight of the appliance to rest on a floor surface while it is being used.

Still another object of the invention is that the appliance be self-powered.

It is an object of the invention to provide improved elements and arrangements thereof by apparatus for the purposes described which is inexpensive, dependable, and fully effective in accomplishing its intended purposes.

These and other objects of the present invention will become readily apparent upon further review of the following specification and drawings.

### BRIEF DESCRIPTION OF THE DRAWINGS

Various objects, features, and attendant advantages of the present invention will become more fully appreciated as the same becomes better understood when considered in conjunction with the accompanying drawings, in which like reference characters designate the same or similar parts throughout the several views, and wherein:

FIG. 1 is a perspective view of a portable irrigating appliance according to at least one aspect of the invention.

FIG. 2 is a perspective view of the portable irrigating appliance of FIG. 1, with a dispensing conduit shown in an extended condition.

FIG. 3 is a diagrammatic cross sectional side view of the portable irrigating appliance of FIG. 1.

FIG. 4 is an exploded perspective view of a nozzle assembly seen at the right side of FIG. 2.

FIG. 5 is a side cross sectional detail view of the nozzle assembly of FIG. 4.

FIG. 6 is an electric schematic diagram of electrical circuitry and components of the portable irrigating appliance, according to at least one aspect of the invention.

FIG. 7 is an electric schematic diagram of electrical circuitry and components of the portable irrigating appliance, according to another aspect of the invention.

### DETAILED DESCRIPTION

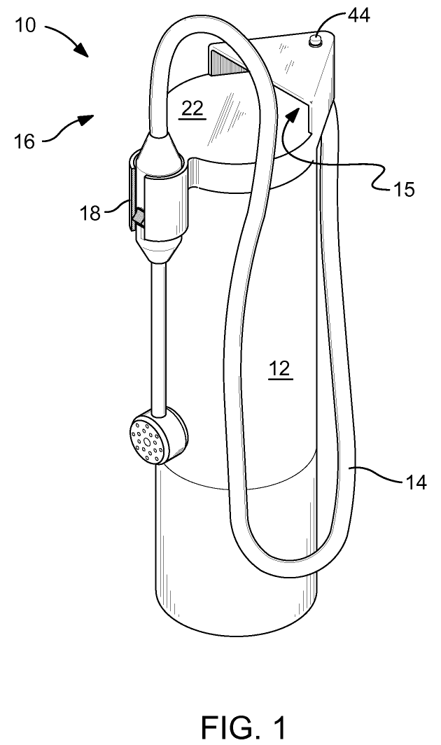

FIG. 1 shows a portable irrigating appliance 10 for enabling personal lavage according to at least one aspect of the invention. The portable irrigating appliance 10 may comprise a housing 12 which serves as a structural platform for supporting other components. The housing 12 may be generally cylindrical and when standing on a horizontal environmental surface (not shown) such as a floor of a building, may be considerably higher than wide or deep. On top of the apparatus there may be a recess 15 to be utilized as a handle for the user to conveniently transport the lavage. Visible at the exterior of the housing 12 are a flexible conduit 14 terminating in a nozzle assembly 16, and a clip 18. The clip 18 is capable of releasably holding the nozzle assembly 14 so that the nozzle assembly 14 is maintained in close proximity to the housing 12. Of course, it would be possible to provide a clip (not shown) which engages the flexible conduit 14 to accomplish a similar function.

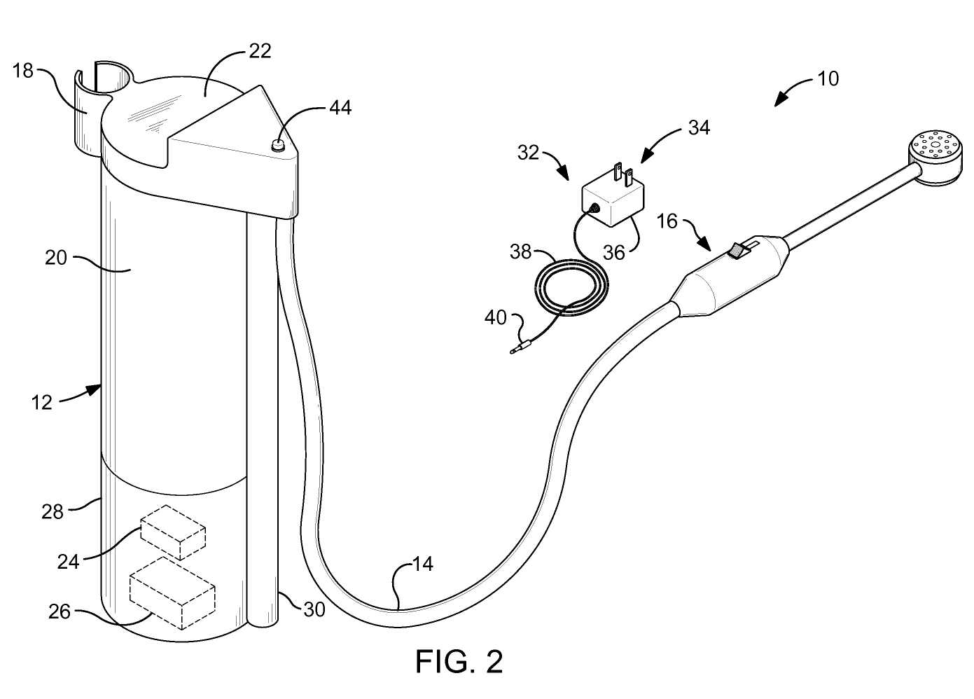

FIG. 2 shows the flexible conduit 14 removed from the clip 18 and extended, as it might be for use for example. The upper portion of the housing 12 defines a reservoir 20 for storing liquids associated with the housing 12.

As employed herein, “associated with” will be understood to signify that the item that is associated with the housing 12 is attached to the housing 12 either internally or externally, in a manner either permanently or integrally connected, or alternatively may be removably attached thereto. In either case, the item associated with the housing 12 is held in close proximity to the housing 12 to the extent that carrying the housing 12 about and changing the location of the housing 12 will move at least part of the item associated therewith. Casual or limited moving or repositioning of the housing 12 will move cause consequential motion of all components of the appliance except the nozzle 62 (described hereinafter) and components of the nozzle 62, since the nozzle 62 is connected to the housing 12 by the flexible conduit 14, which could accommodate minor motion and repositioning without transferring such motion and repositioning to the nozzle 62.

The reservoir 20 may be at least partially light transmissive, so that a water level or liquid level within the reservoir 20 may be readily discerned. The reservoir 20 may include a receptacle portion (not separately shown) and a closure 22 which is manually removable for example to replenish the receptacle of the reservoir 20. The closure 22 may fit by friction for example to the receptacle of the reservoir 20, or in any other suitable way. It should also be noted that the recessed handle 15, may or may not be attached to the closure 22. The receptacle portion of the reservoir 20 may be integral with the housing 12. The housing 12 may extend below the receptacle portion to house additional components such as a pump 24 and a rechargeable battery 26 within a lower portion 28. A vertical riser 30 extends along the housing 12 to enclose a portion of the flexible conduit 14.

The battery 26 may be recharged by a stand-alone battery charger 32 well known type which may comprise a plug 34, a power adapter 36 disposed for example to transform 120 volt AC power to DC power of voltage lower than 120 volts, and a power cord 38 terminating in a terminal 40. The portable irrigating appliance 10 may be provided with a power port (not shown) which is matingly compatible with the terminal 40, to conduct power to the battery 26. Rechargers, such as the battery charger 32, are well known, and their structure and electrical connection ports usable therewith need not be set out in further detail herein.

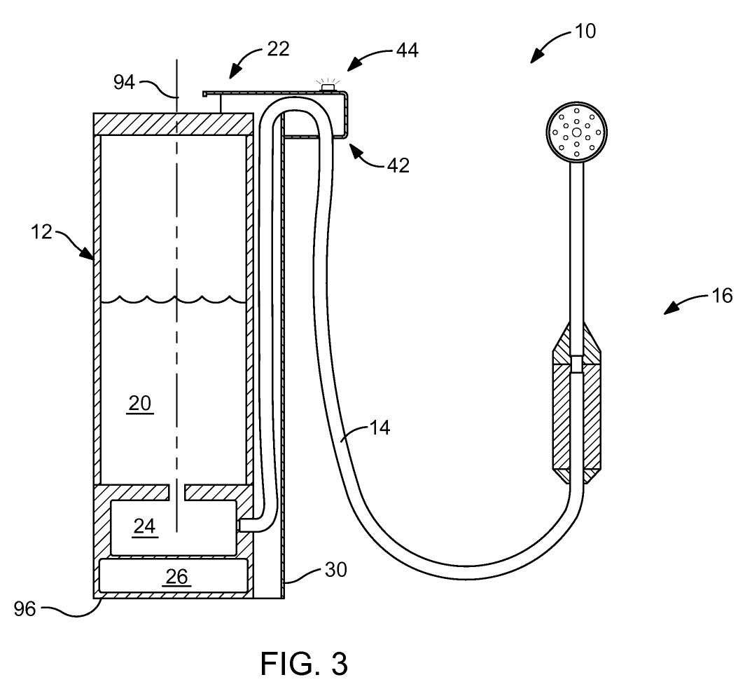

Turning now to FIG. 3, the closure 22 is seen to provide an outlet 42 located so that the flexible conduit 14, which may extend upwardly within the vertical riser 30 from the pump 24, may exit the housing 12 at a point which is relatively high on the housing 12. The closure 22 may also support an indicator light 44 which is disposed to illuminate when the battery charge of the battery 26 falls below a predetermined voltage threshold.

The portable irrigating appliance 10 includes a liquid circuit associated with the housing 12 and disposed in fluid communication with the reservoir 20. The liquid circuit may comprise the reservoir 20, the pump 24, which will be understood to include an electric motor (not separately shown) drivably coupled to the pump 24, the flexible liquid conduit 14, and the nozzle assembly 16.

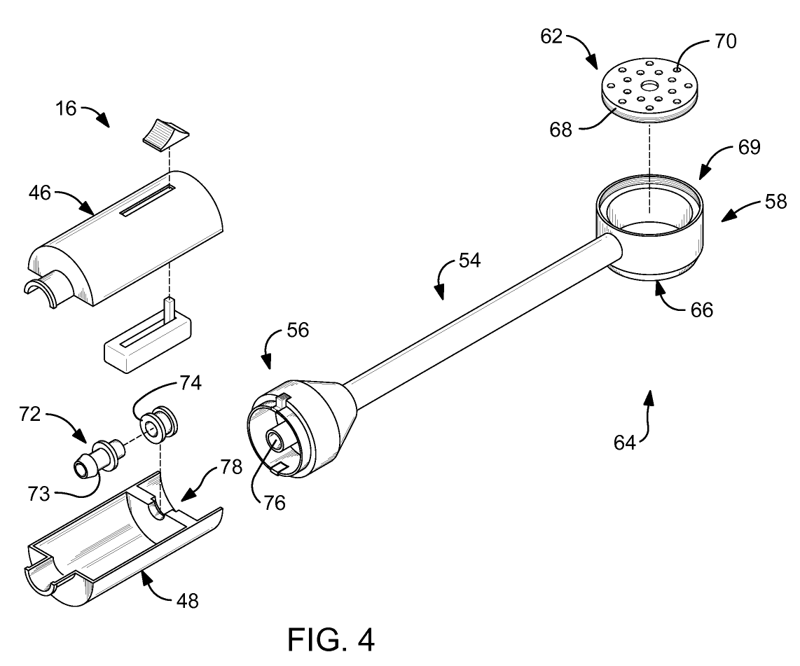

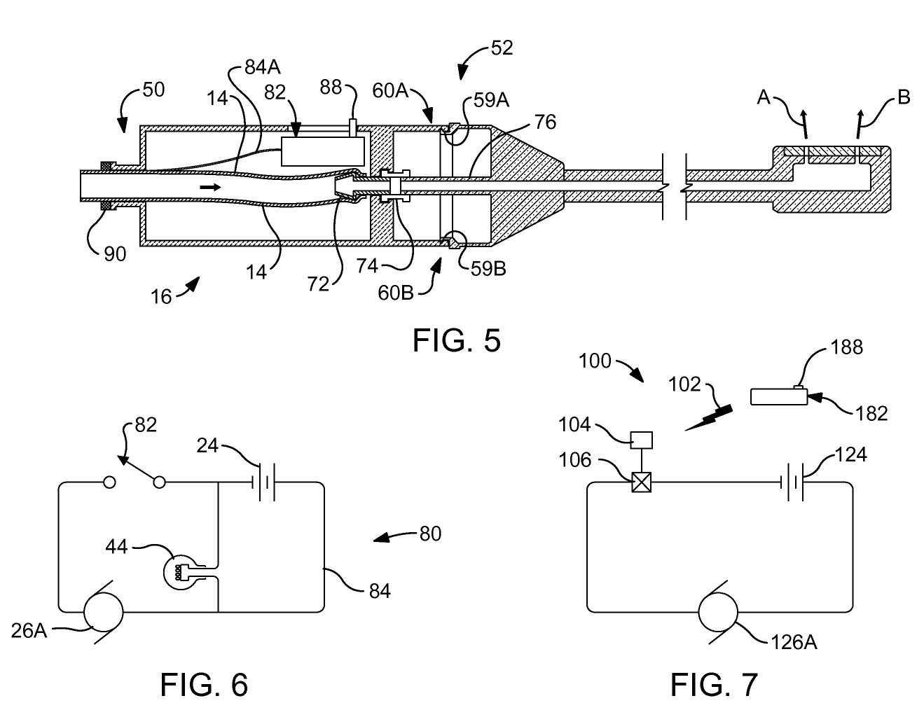

Referring now to FIGS. 4 and 5, the nozzle assembly 16 will be described in greater detail. The nozzle assembly 16 terminates the flexible liquid conduit 14, and may be made from a constituent material displaying form holding rigidity. Form holding signifies that the element so described has sufficient rigidity to maintain its form in the absence of outside forces other than those imposed by the mass and, where provided, elasticity of the constituent materials of the nozzle assembly 16. Rigidity is sufficient to cause at least the exteriorly exposed components to individually or collectively assume the form described herein. It would be possible for the constituent materials to be selectively flexible to deform elastically under outside forces, such as manual handling, but where this is the case, the constituent materials would have sufficient elasticity to reassume the original form when the outside forces are removed.

The nozzle assembly 16 may comprise a two part body comprising a first section 46 and a mating second section 48 (FIG. 4), which collectively include a proximal end 50 (FIG. 5) dimensioned and configured to pass therethrough the flexible conduit 14, and a distal end 52. The distal end 52 may be dimensioned and configured to enable interfitting engagement of a nozzle wand 54 the proximal end 56 of the nozzle wand 54. The proximal end 56 of the nozzle wand 54 may include dogs 59A, 59B which are resilient and tend to expand outwardly after being inwardly compressed as the proximal end 56 is inserted into engagement with the distal end 52 of the two part body. The dogs 59A, 59B engage corresponding notches or depressions 60A, 60B formed in the distal end 52 of the nozzle wand 54 to retain the nozzle wand 54 in place.

Of course, a friction fit, a snap fit, a twisting motion, such as that using helical threads or bayonet connection or the like, and other manual connections, may be provided as alternatives. Also, the relative locations of the actual connecting components, such as the dogs 59A, 59B and their corresponding notches 60A, 6B may be exchanged.

The nozzle wand 54 may be elongated so that the two part body may serve as a handle, with the nozzle 62 spaced conveniently well away from the handle by a wand conduit 58 borne at the distal end 64 of the nozzle wand 54.

The nozzle 62 may comprise an enlarged head 66 having a groove 69 for receiving a removable outlet cover 68. The outlet cover 68 is removable to enable for example cleaning of the nozzle 62, and for ready replacement of the outlet cover 68 itself if desired. The outlet cover 68 may have a plurality of discharge orifices such as the discharge orifice 70 enabling liquids under pressure from the pump 24 to be ejected as a spray (shown representatively as arrows A, B) from the nozzle 62.

The nozzle assembly 16 may house a connection of the flexible conduit 14 to the nozzle wand 54. Connection may be enabled by provision of an interface connector 72 contained within the two part body. The interface connector 72 may have a barbed fitting 73 for securely engaging the flexible conduit 14 and a grommet 74 for engaging a conduit stub 76 formed in the nozzle wand 54. The grommet 74 may be made from a flexible or resilient material such as a silicone rubber for example. The remaining components of the two part body of the nozzle assembly 16 and the nozzle wand 54 may be made from polymeric materials for example, which are substantially rigid so as to enable manual engagement of the nozzle wand 54 to the two part body of the nozzle assembly 16. A seat 78 may be formed in the two part body of the nozzle assembly 16 for receiving and holding the interface connector 72 and the grommet 74 in place.

A power circuit is provided and is connected to the battery 26 as well as to the indicator light 44, as will be further explained hereinafter.

The portable irrigating appliance 10 has a flow controller disposed to control flow from the reservoir 20 to the nozzle 62. The flow controller may comprise the pump 24 and the power system serving and operating the pump 24, or may comprise a valve (not shown) or both. The pump 24 may be operated using a power circuit connected to provide operating power from the battery 26 to the pump 24.

FIG. 6 shows a representative power circuit 80, which may include the battery 24, the motor 26A associated with the pump 26, a switch 82, and the indicator light 44. The switch 82 is in this example a manual switch which controls operating power provided to the pump 26. The power circuit 80 also includes hardwired electrical conductors, shown representatively by reference numeral 84. The electrical conductors 24 will be understood to be potentially discontinuous as they connect intervening components such as the battery 24, the motor 26A, and the switch 82.

The switch 82 may be a variable speed switch located on the nozzle assembly 16, such as a resistor type switch having a slide operator 88 (see FIG. 5). Where the switch 82 is located on the nozzle assembly 16, the electrical conductors 84 may include a hardwired electrical conductor 84A (FIG. 5) extending in close proximity to the flexible liquid conduit 14 from the switch 82 to the housing 12 and further to the battery 24. The flexible liquid conduit 14 and the electrical conductor 84A may be covered by a protective braided sheath 90.

Turning now to FIG. 7, as an alternative to the hardwired circuit of the power circuit 80, a power circuit 100 incorporating a wireless link may be provided to operate a pump, such as the pump 26. In the power circuit 100, a battery such as the battery 124 supplies power to the pump motor such as the pump motor 126A. The battery 124 and the pump motor 126A may be structural and functional counterparts of the respective battery 24 and pump motor 26A, and need not be further detailed herein. A switch 182 may be disposed on a flexible conduit or on a nozzle assembly, which may respectively be the functional counterparts of the flexible conduit 14 and the nozzle assembly 16 for example. This switch 182 may incorporate a radio frequency signal generator (not separately shown), which signal generator may operate according to a protocol such as that of Bluetooth for example, and may generate a radio frequency signal 102 responsive to operation of a manual operator 188, which, apart from controlling a wireless signal generator, may be the functional equivalent of the slide operator 88. The radio frequency signal 102 is received by a transducer 104 which in turn operates a relay 106 to make and break that portion of the circuit 100 connected to the motor 126A. If desired, the transducer 104 and relay 106 may be modified, replaced or both to provide variable speed control of the motor 126A.

Circuitry will be understood to comprise the number of conductors, and specific connection schemes necessary to carry out the described functions, as well as supporting apparatus such as switches, connectors, relays, transducers, circuit breakers and fuses, transformers, and voltage dividers, among others.

Referring again to FIG. 3, the portable irrigating appliance 10 may have the following proportions and characteristics to enable usage as a self-supporting, stand-alone device which may stand erect on a floor or other flat horizontal environmental surface (not shown) with its length projecting upwardly while occupying floor space of diameter not exceeding one third of the height of the portable irrigating appliance 10. To these ends, the housing 12 may have a vertically oriented central axis 94 and a base 96 having a flat bottom surface for stably resting on the flat horizontal environmental surface. The base 96 will be understood to be generally perpendicular to the central axis 94 of the housing 12.

It will be seen that the flat bottom surface of the base 96, the battery 26, the pump 24, and the reservoir 20 are generally aligned vertically along the central axis 94, and are located along the housing 12 in the order just recited. The reservoir 20 is dimensioned and configured such that its length is also generally aligned along the central axis 94.

It should be noted at this point that orientational terms such as vertical and others refer to the referenced drawing figure as viewed by an observer. Therefore, orientational terms must be understood to provide semantic basis for purposes of description, and do not limit the invention, its component parts in any particular way, or usage of any of these in any way.

The present invention is susceptible to modifications and variations which may be introduced thereto without departing from the inventive concepts. For example, although the invention has been described with respect to limited examples, it is to be understood that for example components presented in the singular may be provided in the plural. Where feasible, it would be possible to provide a single component rather than a plurality of components. Also, locations of components as described herein may be modified to suit.

While the present invention has been described in connection with what is considered the most practical and preferred embodiment, it is to be understood that the present invention is not to be limited to the disclosed arrangements, but is intended to cover various arrangements which are included within the spirit and scope of the broadest possible interpretation of the appended claims so as to encompass all modifications and equivalent arrangements which are possible.

I claim:

1. A portable irrigating appliance for enabling personal lavage, comprising:
a housing;
a reservoir for storing liquids associated with the housing, including a receptacle and a manually removable closure;
a liquid circuit associated with the housing and disposed in fluid communication with the reservoir, wherein the liquid circuit comprises a pump, an electric motor drivably coupled to the pump, a flexible liquid conduit, and a nozzle assembly of form holding rigidity disposed to terminate the flexible liquid conduit, and a flow controller disposed to control flow from the reservoir to the nozzle; and
power circuit connected to provide operating power to the pump, including a battery associated with the housing.

2. The portable irrigating appliance of claim 1, wherein the flow controller comprises a switch disposed to control operating power provided to the pump.

3. The portable irrigating appliance of claim 2, wherein the switch is a variable speed switch.

4. The portable irrigating appliance of claim 3, wherein the switch is a resistor type switch having a slide operator, and wherein the power circuit includes a hardwired electrical conductor extending in close proximity to the flexible liquid conduit from the switch to the housing, and wherein the portable irrigating appliance further comprises braided sheath disposed to cover both the flexible conduit and also the hardwired electrical conductor.

5. The portable irrigating appliance of claim 2, wherein the switch is located on the nozzle assembly.

6. The portable irrigating appliance of claim 1, wherein the reservoir is at least partially light transmissive, whereby water level within the reservoir may be readily discerned.

7. The portable irrigating appliance of claim 1, further comprising an indicator light disposed to illuminate when the battery charge falls below a predetermined voltage threshold.

8. The portable irrigating appliance of claim 1, wherein the flow controller includes a wireless signal generator disposed on one of the flexible conduit and the nozzle assembly, and a transducer which receives signals from the wireless signal generator and responsively controls power to the motor of the pump.

9. The portable irrigating appliance of claim 1, wherein the housing further comprises a clip capable of releasably holding one of the flexible conduit and the nozzle assembly.

10. The portable irrigating appliance of claim 1, wherein the nozzle assembly includes a removable outlet cover.

11. The portable irrigating appliance of claim 1, wherein the nozzle assembly comprises:
a two part body comprising a first section and a mating second section, which collectively include a proximal end dimensioned and configured to pass therethrough the flexible conduit, and a distal end dimensioned and configured to enable interfitting engagement;
a nozzle wand comprising an elongated wand conduit having a proximal end dimensioned and configured to interfit with the distal end of the two part body, and a distal end bearing a nozzle; and
an interface connector contained within the two part body, disposed to connect the flexible conduit to the nozzle wand.

12. The portable irrigating appliance of claim 11, wherein the nozzle comprises a removable outlet cover.

13. The portable irrigating appliance of claim 1, wherein the battery is of the rechargeable type, and the portable irrigating appliance comprises a power port arranged to conduct power to the battery, and a stand-alone power adapter comprising a plug, a power adapter disposed to transform 120 volt AC power to DC power of voltage lower than 120 volts, and a power cord terminating in a terminal which is matingly compatible with the power port.

14. The portable irrigating appliance of claim 1, wherein the housing has a central axis and a base having a flat bottom surface for stably resting on a flat horizontal environmental surface, wherein the base is generally perpendicular to the central axis of the housing,
the flat bottom surface, the battery, the pump, and the reservoir are generally aligned along the central axis of the housing, and are located along the housing in the order just recited, and
the reservoir is dimensioned and configured such that its length is generally aligned along the central axis of the housing, whereby
the portable irrigating appliance may stand erect on a floor surface with its length projecting upwardly while occupying floor space of diameter not exceeding one third of the height of the portable irrigating appliance.
